(19) United States
(12) Patent Application Publication
(10) Pub. No.: US 2022/0362360 A1
(43) Pub. Date: Nov. 17, 2022

(54) MRNA FORMULATION

(71) Applicant: eTheRNA immunotherapies NV, Niel (BE)

(72) Inventors: Stefaan De Koker, Niel (BE); Sanne Bevers, Niel (BE); Peter Tomme, Niel (BE)

(21) Appl. No.: 17/604,559

(22) PCT Filed: Apr. 24, 2020
(86) PCT No.: PCT/EP2020/061477

§ 371 Date: Oct. 18, 2021

(30) Foreign Application Priority Data
Apr. 26, 2019 (EP) 19171323.9

(51) Int. Cl. A61K 39/00; A61P 35/00

(52) U.S. Cl. CPC A61K 39/0011; A61P 35/00; A61K 2039/53; A61K 2039/55516; A61K 2039/585

(57) ABSTRACT

The present invention relates to the field of mRNA formulations, and in particular provides a combination of one or more mRNA molecules encoding the functional immunostimulatory proteins CD40L, CD70 and caTLR4; and one or more mRNA molecules encoding an antigen. The combinations of the present invention are in particular characterized in that the mRNA molecules comprise a 5′ CAP-1 structure and may further contain one or more modified nucleosides, such as pseudouridines. The present invention also provides compositions comprising said combinations and uses thereof, in particular in vaccination, and treatment of cell proliferative disorders.

### CROSS REFERENCES TO RELATED APPLICATIONS

This application is a national-stage application under 35 U.S.C. § 371 of International Application No. PCT/EP2020/061477, filed Apr. 24, 2020, which International Application claims benefit of priority to European Patent Application No. 19171323.9, filed Apr. 26, 2019.

### TECHNICAL FIELD

The present invention relates to the field of mRNA formulations, and in particular provides a combination of one or more mRNA molecules encoding the functional immunostimulatory proteins CD40L, CD70 and caTLR4; and one or more mRNA molecules encoding an antigen. The combinations of the present invention are in particular characterized in that the mRNA molecules comprise a 5′ CAP-1 structure and may further contain one or more modified nucleosides, such as pseudouridines. The present invention also provides compositions comprising said combinations and uses thereof, in particular in vaccination, and treatment of cell proliferative disorders.

### BACKGROUND

The magnitude and functional characteristics of T cell responses elicited by mRNA vaccines are governed by the complex interplay between route of administration, delivery vehicle, and the intrinsic properties of the mRNA. The route of administration will determine the biodistribution of mRNA at the organ and cellular level. In this respect, intravenous immunization with mRNA complexed into nanocarriers has emerged as a particularly powerful approach to elicit high magnitude/high quality T cell responses, yet immunogenicity critically depends on the physicochemical properties of the delivery vehicle. Finally, the mRNA format itself is likely to have a huge impact on immunogenicity. Depending on its' 5′Cap structure and on the presence of ssRNA and dsRNA sequence motifs, in vitro transcribed (IVT) RNA can indeed be recognized by various RNA sensors. Whereas triggering of these RNA sensors elicits the necessary cytokine responses to support T cell differentiation, it also elicits antiviral signatures aimed at degrading mRNA and shutting down translation arrest. Hence, in case of mRNA vaccines, innate activation needs to be tightly balanced to enable adequate expression of the mRNA encoded antigen.

Herein, we have addressed the impact of different mRNA formats on the magnitude and functional properties of the vaccine elicited T cell response. Three different mRNA formats were addressed in this study, which differ in their degree of recognition by the innate immune system: 1) ARCA-Capped, non-modified mRNA 2) CleanCapped, non-modified mRNA 3) CleanCapped, modified mRNA. ARCA-Capped mRNA possesses a so-called 5′Cap-0 structure, which stabilizes the mRNA and drives translation initiation, but is recognized by several RNA sensors that drive RNA degradation and translation arrest. Clean-Capped mRNA instead possesses a Cap-1 structure, which renders mRNA invisible for several RNA sensors (MDA-5, RIG-I and IFIT-1). Eukaryotic mRNAs typically have various nucleoside modifications (eg pseudo-uridine, 5 methylcytidine, N1 methylspeudo-uridine) build-in that lower their interaction with Toll-like Receptors (TLRs) 3, 7 and 8. For all three mRNA formats, we assessed the impact of TriMix (mRNA molecules encoding the functional immunostimulatory proteins CD40L, CD70 and caTLR4) on the magnitude of the elicited T cell response as well as on the early cytokine response evoked.

Our studies revealed important differences between the different mRNA formats in terms of T cell response and early inflammatory response. ARCA-capped, unmodified mRNA elicited the most prominent inflammatory response upon intravenous injection. Serum cytokine titers were significantly reduced by switching to Clean-Capped mRNA and further dampened by concomitant substitution of uridine by N1 methylpseudo-uridine. For none of the mRNA formats tested, addition of TriMix exacerbated systemic cytokine responses.

In terms of magnitude of T cell response, no significant differences were observed between the three different mRNA formats in the absence of TriMix (antigen only). Nonetheless, upon addition of TriMix, strong differences in T cell responses emerged between mRNA formats. Whereas TriMix did not improve the magnitude of the T cell response in case of ARCA-Capped, unmodified mRNA, TriMix did strongly enhance the magnitude of the T cell response in case of CleanCapped, unmodified and CleanCapped modified mRNA. Overall, T cell responses were most elevated in case of immunization with E7: TriMix at a ratio of 5:5 and in the Cleancap non-modified and CleanCapped modified mRNA formats, with as high as 80% of all circulating CD8 T cells being specific for E7.

### SUMMARY

In a first aspect, the present invention provides a combination comprising:
one or more mRNA molecules encoding the functional immunostimulatory proteins CD40L, CD70 and caTLR4; and
one or more mRNA molecules encoding an antigen;
wherein at least one of said mRNA molecules is characterized in having a 5′ CAP-1 structure.

In a particular embodiment of the present invention, at least 2, at least 3, or all of said mRNA molecules have a 5′ CAP-1 structure.

In another particular embodiment, said mRNA molecules further comprise at least one modified nucleoside, such as selected from the list comprising pseudouridine, 5-methoxy-uridine, 5-methyl-cytidine, 2-thio-uridine, and N6-methyladenosine.

In a further particular embodiment, said modified nucleoside may be a pseudouridine selected from the list comprising: 4-thio-pseudouridine, 2-thio-pseudouridine, 1-carboxymethyl-pseudouridine, 1-propynyl-pseudouridine, 1-taurinomethyl-pseudouridine, N1-methyl-pseudouridine, 4-thio-1-methyl-pseudouridine, 2-thio-1-methyl-pseudouridine, 1-methyl-1-deaza-pseudouridine, 2-thio-1-methyl-1-deaza-pseudouridine, dihydropseudouridine, 2-thio-dihydropseudouridine, 4-methoxy-pseudouridine, and 4-methoxy-2-thio-pseudouridine. In a very specific embodiment, said at least one modified nucleoside is N1-methyl-pseudouridine.

In another particular embodiment, about 100% of uridines in said mRNA molecules is replaced by N1-methyl-pseudouridine.

The present invention further provides a pharmaceutical composition comprising the combination as defined herein and at least one pharmaceutically acceptable agent.

In another particular embodiment, the combination as defined herein is formulated in nanoparticles, such as lipid nanoparticles or polymeric nanoparticles.

In a further embodiment, the composition as defined herein; is formulated for intravenous, intranodal, intratumoral, subcutaneous, intradermal or intramuscular formulation.

In yet a further specific embodiment, the composition according to this invention may be in the form of a vaccine.

In a particular embodiment of the present invention, said antigen is a cancer antigen.

The present invention further provides a combination, or composition as defined herein for use in human or veterinary medicine; more in particular for use in vaccination and/or for use in the treatment of cell proliferative disorders.

The present invention also provides a method for treating a cell proliferative disorder, said method comprising: administering to a subject in need thereof, a combination or composition as defined herein.

### BRIEF DESCRIPTION OF THE DRAWINGS

With specific reference now to the figures, it is stressed that the particulars shown are by way of example and for purposes of illustrative discussion of the different embodiments of the present invention only. They are presented in the cause of providing what is believed to be the most useful and readily description of the principles and conceptual aspects of the invention. In this regard no attempt is made to show structural details of the invention in more detail than is necessary for a fundamental understanding of the invention. The description taken with the drawings making apparent to those skilled in the art how the several forms of the invention may be embodied in practice.

FIG. 1: Impact of mRNA format and TriMix on systemic inflammatory responses. Serum cytokine titers measured at 6 hours following intravenous injection of mice with the indicated mRNA LNPs. E7/TriMix mRNA was encapsulated at ratio's 10:0, 7.5:2.5, 5:5 into LNPs (50/10/39.5/0.5). mRNA was either ARCA capped without nucleoside modifications (ARCA/non-mod), CleanCapped without nucleoside modifications (CleanCap/non-mod) or CleanCap and containing N1methylpseudo-uridine (CleanCap/mod). n=6; Two-way ANOVA analysis. ns=non-significant; *p<0.05; **p<0.01; ***p<0.001****p<0.0001.

FIG. 2: Kinetics of inflammatory responses to CleanCap/modified mRNA LNPs. E7 and TriMix mRNA were encapsulated at the indicated ratios into LNPs and administrated intravenously at a total mRNA dose of 10 μg. All mRNA contained a CleanCap and a 100% uridine substitution by N1methylpseudo-uridine. Serum cytokines were measured at 6 hours and 24 hours post injection.

FIGS. 3A and 3B: FIG. 3A) Impact of mRNA format on the magnitude of the E7-specific T cell response upon immunization with E7/TriMix mRNA LNPs at E7/TriMix ratios of 10:0 (no TriMix) and 5:5. FIG. 3B) Impact of TriMix on the magnitude of the E7-specific T cell response in case of immunization with ARCA/non-mod mRNA and CleanCap mod mRNA at the indicated E7 to TriMix ratios. Two-way ANOVA analysis followed by Tukey's multiple comparisons test. ns =non-significant; *p<0.05**p<0.01***p<0.001.

FIG. 4: Magnitude of the IFN-γ T cell response as assessed by ELISPOT after the 3th immunization with E7 E7/TriMix mRNA LNPs at E7/TriMix ratios of 10:0 (no TriMix) and 5:5. mRNA was either ARCA capped without nucleoside modifications (ARCA/non-mod), CleanCapped without nucleoside modifications (CleanCap/non-mod) or CleanCap and containing N1methylpseudo-uridine (CleanCap/mod). n=6; Two-way ANOVA analysis. ns=non-significant; *p<0.05; **p<0.01.

FIGS. 5A and 5B: FIG. 5A) Weight of mice expressed as % of initial body weight upon injection of mice with E7 mRNA LNPs with the indicated mRNA formats. LNPs were intravenously injected at days 0, 7 and 14. FIG. 5B) Weight of mice expressed as % of initial body weight upon injection of mice with LNPs at the indicated E7/TriMix mRNA LNPs for respectively ARCA, non-mod mRNA, CleanCap, non-mod mRNA and CleanCap, mod mRNA.

FIG. 6: ALT/AST levels in serum of mice after the third injection with PBS or the indicated mRNA LNPs. Two-way ANOVA analysis followed by Tukey's multiple comparisons test. ns=non-significant; *p<0.05**p<0.01***p<0.001.

### DETAILED DESCRIPTION

As already detailed herein above, the present invention provides a combination comprising:
one or more mRNA molecules encoding the functional immunostimulatory proteins CD40L, CD70 and caTLR4; and
one or more mRNA molecules encoding an antigen;
wherein at least one of said mRNA molecules is characterized in having a 5′ CAP-1 structure.

Throughout the invention, the term “TriMix” stands for a mixture of mRNA molecules encoding CD40L, CD70 and caTLR4 immunostimulatory proteins. The use of the combination of CD40L and caTLR4 generates mature, cytokine/chemokine secreting DCs, as has been shown for CD40 and TLR4 ligation through addition of soluble CD40L and LPS. The introduction of CD70 into the DCs provides a co-stimulatory signal to CD27⁺ naive T-cells by inhibiting activated T-cell apoptosis and by supporting T-cell proliferation. As an alternative to caTLR4, other Toll-Like Receptors (TLR) could be used. For each TLR, a constitutive active form is known, and could possibly be introduced into the DCs in order to elicit a host immune response. In our view however, caTLR4 is the most potent activating molecule and is therefore preferred.

The mRNA or DNA used or mentioned herein can either be naked mRNA or DNA, or protected mRNA or DNA. Protection of DNA or mRNA increases its stability, yet preserving the ability to use the mRNA or DNA for vaccination purposes. Non-limiting examples of protection of both mRNA and DNA can be: liposome-encapsulation, protamine-protection, (Cationic) Lipid Lipoplexation, lipidic, cationic or polycationic compositions, Mannosylated Lipoplexation, Bubble Liposomation, Polyethylenimine (PEI) protection, liposome-loaded microbubble protection etc..

While the present invention is particularly suitable for use in connection with tumor-specific antigens, it may also be suitably used in connection with other types of target-specific antigens.

The term “target” used throughout the description is not limited to the specific examples that may be described herein. Any infectious agent such as a virus, a bacterium or a fungus may be targeted. In addition any tumor or cancer cell may be targeted. The term “target-specific antigen” used throughout the description is not limited to the specific examples that may be described herein. It will be clear to the skilled person that the invention is related to the induction of immunostimulation in APCs, regardless of the target-specific antigen that is presented. The antigen that is to be presented will depend on the type of target to which one intends to elicit an immune response in a subject. Typical examples of target-specific antigens are expressed or secreted markers that are specific to tumor, bacterial and fungal cells or to specific viral proteins or viral structures. Without wanting to limit the scope of protection of the invention, some examples of possible markers are listed below.

The terms “neoplasms”, “cancer” and/or “tumor” used throughout the description are not intended to be limited to the types of cancer or tumors that may have been exemplified. The term therefore encompasses all proliferative disorders such as neoplasma, dysplasia, premalignant or precancerous lesions, abnormal cell growths, benign tumors, malignant tumors, cancer or metastasis, wherein the cancer may be selected from the group of: leukemia, non-small cell lung cancer, small cell lung cancer, CNS cancer, melanoma, ovarian cancer, kidney cancer, prostate cancer, breast cancer, glioma, colon cancer, bladder cancer, sarcoma, pancreatic cancer, colorectal cancer, head and neck cancer, liver cancer, bone cancer, bone marrow cancer, stomach cancer, duodenum cancer, oesophageal cancer, thyroid cancer, hematological cancer, and lymphoma. Specific antigens for cancer can e.g. be MelanA/MART1, Cancer-germline antigens, gp100, Tyrosinase, CEA, PSA, Her-2/neu, survivin, telomerase.

In a preferred embodiment of the vaccine of the invention, the mRNA or DNA molecule(s) encode(s) the CD40L and CD70 immunostimulatory proteins. In a particularly preferred embodiment of the vaccine of the invention, the mRNA or DNA molecule(s) encode(s) CD40L, CD70, and caTLR4 immunostimulatory proteins.

Said mRNA or DNA molecules encoding the immunostimulatory proteins can be part of a single mRNA or DNA molecule. Preferably, said single mRNA or DNA molecule is capable of expressing the two or more proteins simultaneously. In one embodiment, the mRNA or DNA molecules encoding the immunostimulatory proteins are separated in the single mRNA or DNA molecule by an internal ribosomal entry site (IRES) or a self-cleaving 2a peptide encoding sequence.

In some preferred embodiments, the mRNA used in the methods of the present invention has a 5′ cap structure with a so-called CAP-1 structure (CleanCap), meaning that the 2′ hydroxyl of the ribose in the penultimate nucleotide with respect to the cap nucleotide is methylated, such as illustrated below:

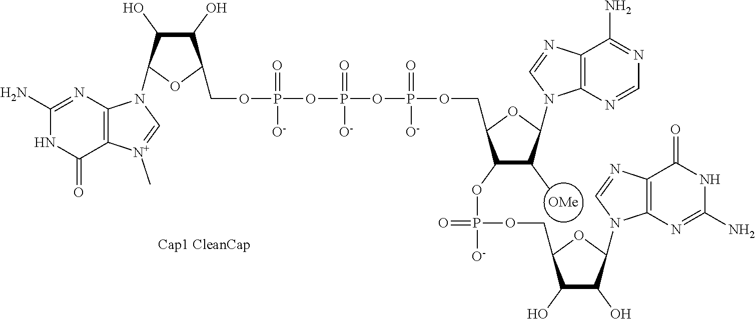

In another particular embodiment, two, three, four,... or all of the used mRNA molecules of the present invention have a 5′ cap structure with a so-called CAP-1 structure.

In a further embodiment, one or more of the mRNA molecules of the present invention may further comprise at least one modified nucleoside. In another particular embodiment, two, three, four, . . . or all of the used mRNA molecules of the present invention have at least one modified nucleoside.

In another particular embodiment of the present invention, said mRNA molecules further comprise at least one modified nucleoside, such as selected from the list comprising pseudouridine, 5-methoxy-uridine, 5-methyl-cytidine, 2-thio-uridine, and N6-methyladenosine.

In a particular embodiment of the present invention, said at least one modified nucleoside may be a pseudouridine, such as selected from the list 4-thio-pseudouridine, 2-thio-pseudouridine, 1-carboxymethyl-pseudouridine, 1-propynyl-pseudouridine, 1-taurinomethyl-pseudouridine, N1-methyl-pseudouridine, 4-thio-1-methyl-pseudouridine, 2-thio-1-methyl-pseudouridine, 1-methyl-1-deaza-pseudouridine, 2-thio-1-methyl-1-deaza-pseudouridine, dihydropseudouridine, 2-thio-dihydropseudouridine, 4-methoxy-pseudouridine, and 4-methoxy-2-thio-pseudouridine. In a very specific embodiment, said at least one modified nucleoside is N1-methyl-pseudouridine.

Alternative nucleoside modifications which are suitable for use within the context of the invention, include: pyridin-4-one ribonucleoside, 5-aza-uridine, 2-thio-5-aza-uridine, 4-thio-pseudouridine, 2-thio-pseudouridine, 5-hydroxyuridine, 3-methyluridine, 5-carboxymethyl-uridine, 1-carboxymethyl-pseudouridine, 5-propynyl-uridine, 1-propynyl-pseudouridine, 5-taurinomethyluridine, 1-taurinomethyl-pseudouridine, 5-taurinomethyl-2-thio-uridine, 1-taurinomethyl-4-thio-uridine, 5-methyl-uridine, 1-methyl-pseudouridine, 4-thio-1-methyl-pseudouridine, 2-thio-1-methyl-pseudouridine, 1-methyl-1-deaza-pseudouridine, 2-thio-1 -methyl-1-deaza-pseudouridine, dihydrouridine, dihydropseudouridine, 2-thio-dihydrouridine, 2-thio-dihydropseudouridine, 2-methoxyuridine, 2-methoxy-4-thio-uridine, 4-methoxy-pseudouridine, and 4-methoxy-2-thio-pseudouridine. In some embodiments, the mRNA comprises at least one nucleoside selected from the group consisting of 5-aza-cytidine, pseudoisocytidine, 3-methyl-cytidine, N4-acetylcytidine, 5-formylcytidine, N4-methylcytidine, 5-hydroxymethylcytidine, 1-methyl-pseudoisocytidine, pyrrolo-cytidine, pyrrolo-pseudoisocytidine, 2-thio-cytidine, 2-thio-5-methyl-cytidine, 4-thio-pseudoisocytidine, 4-thio-1 -methyl-pseudoisocytidine, 4-thio-1-methyl-1-deaza-pseudoisocytidine, 1-methyl-1 -deaza-pseudoisocytidine, zebularine, 5-aza-zebularine, 5-methyl-zebularine, 5-aza-2-thio-zebularine, 2-thio-zebularine, 2-methoxy-cytidine, 2-methoxy-5-methyl-cytidine, 4-methoxy-pseudoisocytidine, and 4-methoxy-1-methyl-pseudoisocytidine. In some embodiments, the mRNA comprises at least one nucleoside selected from the group consisting of 2-aminopurine, 2,6-diaminopurine, 7-deaza-adenine, 7-deaza-8-aza-adenine, 7-deaza-2-aminopurine, 7-deaza-8-aza-2-aminopurine, 7-deaza-2,6-diaminopurine, 7-deaza-8-aza-2,6-diaminopurine, 1-methyladenosine, N6-isopentenyladenosine, N6-(cis-hydroxyisopentenyl)adenosine, 2-methylthio-N6-(cis-hydroxyisopentenyl) adenosine, N6-glycinylcarbamoyladenosine, N6-threonylcarbamoyladenosine, 2-methylthio-N6-threonyl carbamoyladenosine, N6,N6-dimethyladenosine, 7-methyladenine, 2-methylthio-adenine, and 2-methoxy-adenine. In some embodiments, mRNA comprises at least one nucleoside selected from the group consisting of inosine, 1-methyl-inosine, wyosine, wybutosine, 7-deaza-guanosine, 7-deaza-8-aza-guanosine, 6-thio-guanosine, 6-thio-7-deaza-guanosine, 6-thio-7-deaza-8-aza-guanosine, 7-methyl-guanosine, 6-thio-7-methyl-guanosine, 7-methylinosine, 6-methoxy-guanosine, 1-methylguanosine, N2-methylguanosine, N2,N2-dimethylguanosine, 8-oxo-guanosine, 7-methyl-8-oxo-guanosine, 1-methyl-6-thio-guaiguanosine, and N2,N2-dimethyl-6-thio-guanosine.

The mRNA molecules used in the present invention may contain one or more modified nucleotides, in particular embodiment, at least 10%, 20%, 30%, 40%, 50%, 60%, 70%, 80%, 90% or 100% of a particular type of nucleotides may be replaced by a modified one. It is also not excluded that different nucleotide modifications are included within the same mRNA molecule. In a very specific embodiment of the present invention, about 100% of uridines in said mRNA molecules is replaced by N1-methyl-pseudouridine.

The present invention further provides a pharmaceutical composition comprising the combination as defined herein and at least one pharmaceutically acceptable agent.

In a specific embodiment, one or more of said mRNA molecules of the present invention may further contain a translation enhancer and/or a nuclear retention element. Suitable translation enhancers and nuclear retention elements are those described in WO2015071295.

In a further embodiment of the present invention, said one or more mRNA molecules are formulated for parenteral administration; more in particular for intravenous, intranodal, intratumoral, subcutaneous, intradermal or intramuscular formulation.

In yet a further specific embodiment, said mRNA molecules are formulated for intranodal or intratumoral administration, and are in the form of naked mRNA molecules in a suitable injection buffer, such as a Ringer Lactate buffer.

The present invention also provides a combination or composition as defined herein; wherein one or more of said mRNA molecules are encompassed in nanoparticles.

As used herein, the term “nanoparticle” refers to any particle having a diameter making the particle suitable for systemic, in particular intravenous administration, of, in particular, nucleic acids, typically having a diameter of less than 1000 nanometers (nm).

In a specific embodiment of the present invention, the nanoparticles are selected from the list comprising: lipid nanoparticles and polymeric nanoparticles.

A lipid nanoparticle (LNP) is generally known as a nanosized particle composed of a combination of different lipids. While many different types of lipids may be included in such LNP, the LNP's of the present invention may for example be composed of a combination of an ionisable lipid, a phospholipid, a sterol and a PEG lipid.

A polymeric nanoparticle can typically be a nanosphere or a nanocapsule. Two main strategies are used for the preparation of polymeric nanoparticles, i.e. the “top-down” approach and the “bottom-up” approach. In the top-down approach, a dispersion of preformed polymers produces polymeric nanoparticles, whereas in the bottom-up approach, polymerization of monomers leads to the formation of polymeric nanoparticles. Both top-down and bottom-up methods use synthetic polymers/monomers like poly(d,1-lactide-co-glycolide), poly(ethyl cyanoacrylate), poly(butyl cyanoacrylate), poly(isobutyl cyanoacrylate), and poly(isohexyl cyanoacrylate); stabilizers like poly(vinyl alcohol) and didecyldimethylammonium bromide; and organic solvents like dichloromethane and ethyl acetate, benzyl alcohol, cyclohexane, acetonitrile, acetone, and so on. Recently the scientific community has been trying to find alternatives for synthetic polymers by using natural polymers and synthesis methods with less toxic solvents.

The present invention also provides the combinations and vaccines as defined herein for use in human or veterinary medicine, in particular for use in the treatment of cell proliferative disorders, more in particular for use in eliciting an immune response towards a tumor in a subject.

Finally, the present invention provides a method for the treatment of a cell proliferative disorder comprising the steps of administering to a subject in need thereof a combination or vaccine of the present invention.

The compositions may also be of value in the veterinary field, which for the purposes herein not only includes the prevention and/or treatment of diseases in animals, but also—for economically important animals such as cattle, pigs, sheep, chicken, fish, etc.—enhancing the growth and/or weight of the animal and/or the amount and/or the quality of the meat or other products obtained from the animal.

The invention will now be illustrated by means of the following synthetic and biological examples, which do not limit the scope of the invention in any way.

### EXAMPLES

### Materials and Methods

### Mice

Female C57BL/6 Mice were purchased from Charles River Laboratories (France) and housed in individually vented cages with standard bedding material and cage enrichment. The animals were maintained and treated in accordance to the institutional (Vrije Universiteit Brussel) and European Union guidelines for animal experimentation. Mice had ad libitum access to food and water. Experiments started when mice were 8 weeks old. Mice (n=6/group) received 3 intravenous injections via the tail vein with 10 ug mRNA in LNPs (in a volume of 200μL) at days 0, 7 and 14. Control mice were injected with 200 μl of PBS at identical time intervals. Weight of mice was monitored every 2 days.

mRNA Synthesis and Purification

E7 mRNA was prepared by eTheRNA by in vitro transcription (IVT) from the eTheRNA plasmid pEtherna-v2. The sequence encoding the HPV16-E7 protein was cloned in-frame between the signal sequence and the transmembrane and cytoplasmic regions of human DC-LAMP. This chimeric gene was cloned in the pEtherna-v2 plasmid (WO2015071295) that was enriched with a translation enhancer at the 5′ end and an RNA stabilizing sequence at the 3′ end. CD40L, caTLR4 and CD70 mRNA (TriMix components) were cloned in the pEtherna-v2 plasmid. After IVT, dsRNA was removed by cellulose purification. Cellulose powder was purchased from Sigma and washed in 1xSTE (Sodium Chloride-Tris-EDTA) buffer with 16% ethanol. IVT mRNA (in 1xSTE buffer with 16% ethanol) was added to the washed cellulose pellet and shaken at room temperature for 20 minutes. This solution is then brought over a vacuum filter (Corning). The eluate contains the ssRNA fraction and was used for all experiments. mRNA quality was monitored by capillary gel electrophoresis (Agilent, Belgium).

Generation of mRNA Lipid-Based Nanoparticles

Lipid based nanoparticles are produced by microfluidic mixing of an mRNA solution in malic acid buffer (20 mM malic acid (Sigma), 30 mM NaCl (Sigma), pH3) and lipid solution in a 2:1 volume ratio at a speed of 9 mL/min using the NanoAssemblr Benchtop (Precision Nanosystems). The lipid solution contained a mixture of Coatsome-EC (NOF corporation), DOPE (Avanti), Cholesterol (Sigma) and DMG-PEG2000 (Sunbright GM-020, NOF corporation) in a molar ratio of 50/10/39.5/0.5 respectively. LNPs were dialysed against PBS (100 times more PBS volume than LNP volume) using slide-a-lyzer dialysis casettes (20K MWCO, 3 mL, ThermoFisher). Size and polydispersity were measured by dynamic light scattering with a Zetasizer Nano (Malvern).

### Assessment Of Cytokine Titers

6 and 24 hours after the first mRNA LNP administration, 50 uL of blood from treated and control mice was collected in tubes containing a clotting activator (ref 41.1500.005, Sarstedt). Collection tubes were centrifuged (5 min, 10000 g) and serum was transferred to an Eppendorf tube and stored at −20° C. until use. For assessment of titers of the cytokines IFN-a, IFN-g, MCP-1, IL-6, IL-12, RANTES, G-CSF and IP-10, serum was thawed on ice and used in a ProcartaPlex Immunoassay (ThermoFisher Scientific). The assay was performed according to protocol. Serum samples were diluted 3 times in universal assay buffer (included in ProcartaPlex kit) and incubated with fluorescently labelled beads for 120 minutes. Read-out of Procartaplex assay was done on a MagPix instrument (Luminex).

### ALT/AST ELISA

24 hours after the third mRNA LNP administration, 50 uL of blood from treated and control mice was collected in tubes containing a clotting activator (ref 41.1500.005, Sarstedt). Collection tubes were centrifuged (5 min, 10000 g) and serum was transferred to an Eppendorf tube and stored at −20° C. until use. For assessment of serum titers of alanine transaminase 1 (ALT) and aspartate transaminase (AST), serum was thawed on ice and used in an ALT1 ELISA (Abbexa ref. abx570182) and an AST ELISA (Abbexa ref. abx255199). The assays were performed according to the kit protocols with only one deviation; only half of the prescribed volume of serum was added to the plates because of the limiting nature of this type of sample. Serum samples were diluted 5 times in sample diluent buffer included in the kits. Read-out of the ELISAs was done on a SpectraMax M3 plate reader (Molecular Devices).

### Flow Cytometry

Number of E7-specific T Cells

Blood was collected from treated and control mice on days 6, 13 and 19. Red blood cells were lysed and remaining the white blood cells were stained with APC labelled E7_{(RAHYNIVTF)}-tetramer (SEQ ID NO:1) according to the manufacturer's instructions (MBL International). Excess tetramer was washed away and an antibody mixture for surface molecules (listed in table 1) was added to the cells and incubated for 30 minutes at 4° C. Data was acquired on an LSR Fortessa cytometer and analyzed with Flow Jo Software.

TABLE 1
List of antibodies used for flow cytometry analysis of
number of E7-specific T cells.

| Antibody | Fluorochrome | Clone | Company |
|---|---|---|---|
| Viability dye | Zombie Aqua | n.a. | BioLegend |
| CD3 | PerCPeF710 | 17A2 | eBioscience (Thermo Fisher) |
| CD8 | V450 | 53-6.7 | BD Horizon |

IFN-γ ELISpot assessment

A murine IFN-γ ELISpot kit (Diaclone, USA) was used. Microtiter plates (96-well, Diaclone, USA) were coated overnight with anti-IFN-γ capture antibody according to the manufacturer's instructions and aspecific binding was blocked with 10% FBS containing RPMI cell line culture medium for 2 h at 37° C. On day 20, mice were euthanized and their spleens were harvested. Single cell suspensions of splenocytes were prepared and seeded into microtiter plates at 10000 cells/well, with or without peptide stimulation. 5 ug/mL of E7 peptide was used for stimulation in the dedicated wells. As a positive control, T cells were stimulated with anti-CD3/anti-CD28 beads. Cells were incubated for 24 h. Thereafter, cells were washed and spots were developed according to manufacturer's instructions (Diaclone). Spots were counted using an AID ELISpot reader (Autoimmun Diagnostika GmbH, Strassberg, Germany).

### Results And Discussion

### LNP Formulation wnd Physicochemical Properties

All mRNA LNPs were generated by microfluidic mixing on a NanoAssemblr (PNI). In brief, an ethanolic lipid mix composed of SS-EC, DOPE, cholesterol and DMG-PEG2000 was mixed with an acidic solution of the mRNAs of interest as explained in detail in the materials and methods section. The molar % ratio for the constituent lipids is 50% CoatsomeSS-EC, 10% DOPE, 39.5% Cholesterol and 0.5% DMG-PEG2000. mRNA LNPs were subsequently dialyzed to PBS and size and polydispersity (PDI) of all mRNA LNPs were measured by Dynamic Light Scattering (DLS). As indicated in table 2, all mRNA LNPs were of similar size and PDI, regardless of mRNA format and E7: TriMix ratio.

TABLE 2
Size and PDI (polydispersity) of lipid-based nanoparticles
used for intravenous immunizations

| | Immunization 1 | | Immunization 2 | | Immunization 3 | | Average | |
|---|---|---|---|---|---|---|---|---|
| | Size | | Size | | Size | | Size | |
| | (nm) | PDI | (nm) | PDI | (nm) | PDI | (nm) | PDI |
| 10 ug E7, | 130 | 0.112 | 115 | 0.087 | 109 | 0.089 | 118 | 0.096 |
| ARCA, non-mod | | | | | | | | |
| 10 ug E7, | 129 | 0.121 | 117 | 0.088 | 103 | 0.082 | 116 | 0.097 |
| CleanCap, non-mod | | | | | | | | |
| 10 ug E7, | 119 | 0.097 | 115 | 0.085 | 110 | 0.085 | 115 | 0.089 |
| CleanCap, mod | | | | | | | | |
| 7.5/2.5 ug E7/TriMix, | 125 | 0.090 | 109 | 0.102 | 104 | 0.094 | 113 | 0.095 |
| ARCA, non-mod | | | | | | | | |
| 7.5/2.5 ug E7/TriMix, | 116 | 0.114 | 111 | 0.111 | 108 | 0.093 | 111 | 0.106 |
| CleanCap, non-mod | | | | | | | | |
| 7.5/2.5 ug E7/TriMix, | 118 | 0.104 | 114 | 0.088 | 116 | 0.096 | 116 | 0.096 |
| CleanCap, mod | | | | | | | | |
| 5/5 ug E7/TriMix, | 117 | 0.118 | 119 | 0.125 | 115 | 0.137 | 117 | 0.127 |
| ARCA, non-mod | | | | | | | | |
| 5/5 ug E7/TriMix, | 115 | 0.076 | 117 | 0.113 | 113 | 0.088 | 115 | 0.092 |
| CleanCap, non-mod | | | | | | | | |
| 5/5 ug E7/TriMix, | 114 | 0.104 | 116 | 0.117 | 108 | 0.058 | 112 | 0.093 |
| CleanCap, mod | | | | | | | | |
| AVERAGE | 120 | 0.104 | 115 | 0.102 | 110 | 0.091 | 115 | 0.099 |

mRNA Format Determines Inflammatory Responses to E7/Trimix mRNA LNPS

mRNAs were either co-transcriptionally capped with an ARCA-Cap or with a CleanCap. No nucleoside modifications were incorporated into the ARCA Cap mRNA (ARCA, non-mod mRNA). CleanCap mRNA either did not contain modified nucleosides (CleanCap, non-mod mRNA) or displayed a 100% substitution of uridine by Nlmethylpseudo-uridine (CleanCap, mod mRNA). To address the impact of mRNA format and of TriMix on the inflammatory response, E7 mRNA was mixed with TriMix at the indicated ratio's and encapsulated into LNPs. C57BL/6 mice received a total dose of 10 μg mRNA administrated by the tail vein. Serum samples were collected at 6 hours and 24 hours post injection and analyzed for IFN-a, IFN-g, IL-6, MCP-1, G-CSF and RANTES.

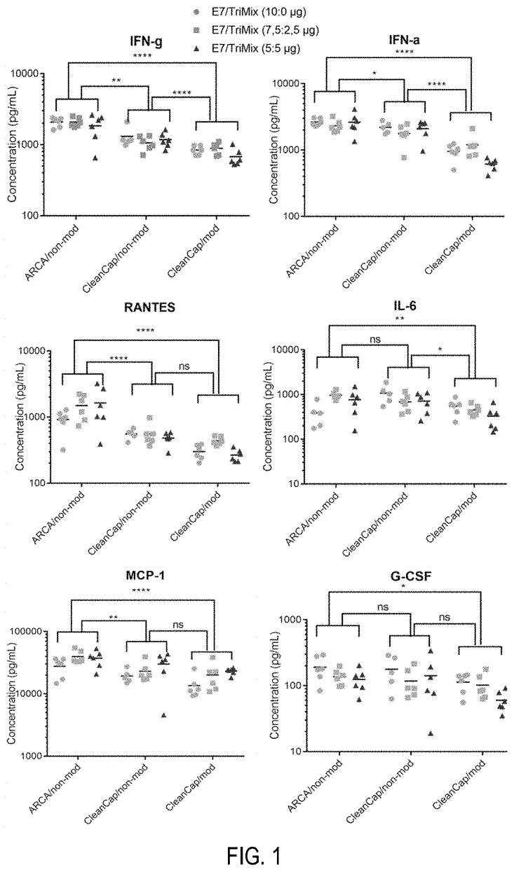

As can be appreciated from FIG. 1, mere replacement of ARCA by CleanCap without incorporation of N1 methylpseudo-uridine resulted in a significant reduction of several (IFN-a, IFN-g, RANTES and MCP-1) but not all cytokines addressed (IL-6, G-CSF). Concomitant replacement of uridine by N1 methylpseudo-uridine (CleanCap, mod) further dampened the inflammatory response, with all cytokines now being significantly reduced in comparison to the ARCA, non-mod mRNA. These data are in line with the differential sensing of ARCA versus CleanCapped mRNA by cytosolic RNA sensors and with the reduced recognition of N1 methylpseudo-uridine mRNA by endosomal TLRs. Surprisingly, partial replacement of E7 mRNA by TriMix mRNA did not elevate serum titers of inflammatory cytokines.

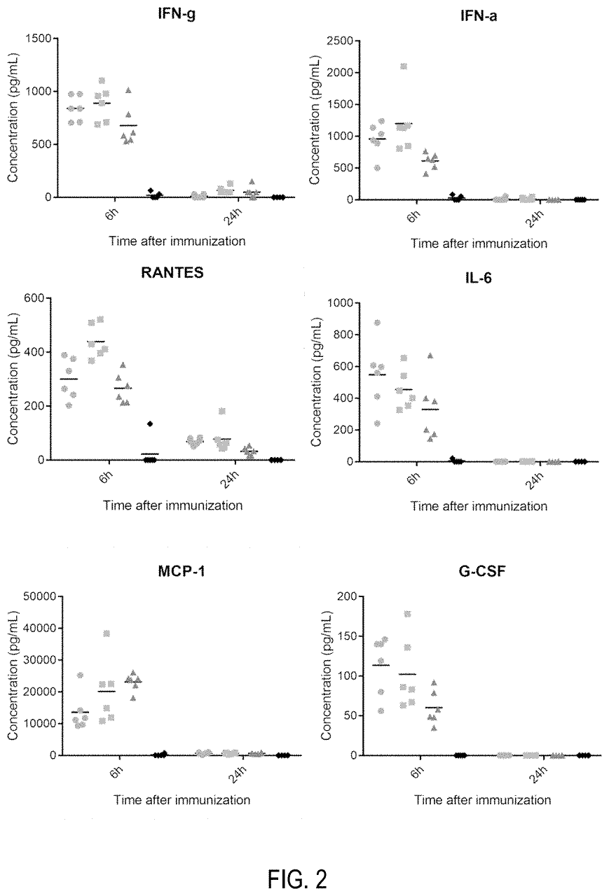

We next addressed the kinetics of the inflammatory response of intravenously injected mRNA LNPs containing CleanCapped, N1 methylpseudo-uridine modified mRNA (FIG. 2). Whereas all cytokines showed significant elevation at 6 hours post injection, cytokine titers had returned to near-baseline at 24 hours post injection, which clearly indicate that cytokine responses to CleanCap/mod mRNA LNPs are transient and not fuel cytokine release syndromes.

mRNA Format Impacts the Magnitude and Functionality of the Antigen-Specific T Cell Responses

To assess the impact of the mRNA format on the magnitude and functional characteristics of the T cell response, E7 mRNA was mixed with TriMix mRNA at the indicated ratios for the different mRNA formats. Mice were immunized at days 0, 7 and 14 with the respective mRNA LNPs at a total mRNA dose of 10 μg. At days 6, 13 and 19, PBMCs were collected and stained by flow cytometry to assess the percentages of E7-specific CD8 T cells.

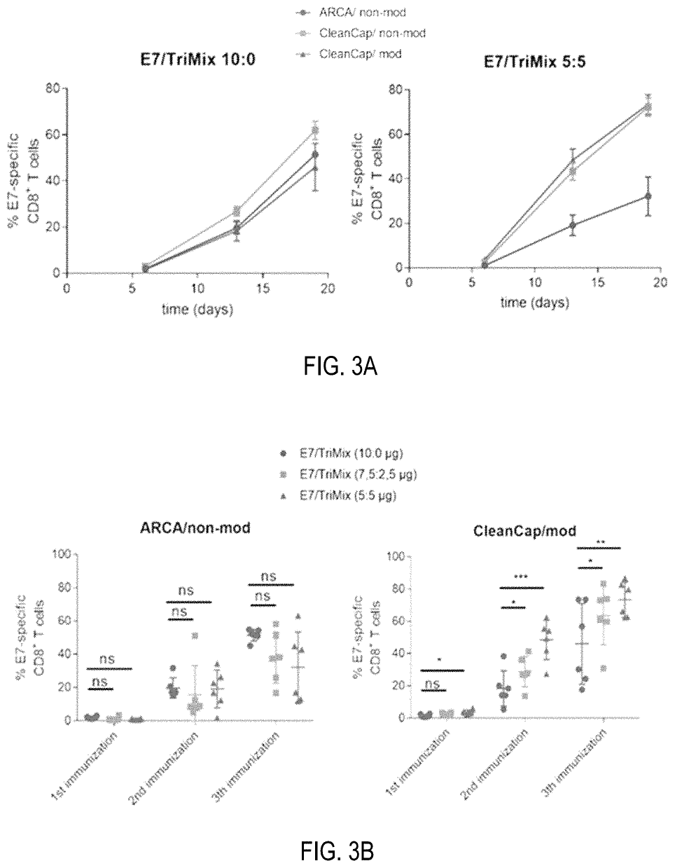

As can be appreciated from FIGS. 3A and 3B, intravenous immunization with mRNA LNPs elicited a robust E7-specific T cell response, with the highest percentages of E7-specific T cells being elicited in the CleanCap/non-mod mRNA LNP and the CleanCap/mod mRNA LNPs treatment groups at an E7: TriMix ratio 5:5.

In the absence of TriMix mRNA (E7: TriMix 10:0), no significant differences were observed in the magnitude of the elicited T cell response between the three mRNA formats. Nonetheless, when half of the E7 mRNA was substituted by TriMix (ratio 5:5), mice immunized with CleanCap/non-mod mRNA and CleanCap/mod mRNA showed significantly higher levels of E7-specific T cells compared to mice immunized with ARCA/non-mod mRNA (FIG. 3A). The impact of TriMix hence clearly dependent on the mRNA format, with TriMix showing no benefit in case of ARCA/non-mod mRNA yet showing a highly significant benefit in case of CleanCap/mod mRNA.

In case of CleanCap/mod mRNA, E7/TriMix at a 5:5 ratio appeared to evoke stronger T cell responses compared to the 7.5:2.5 ratio, warranting additional studies to further optimize the ratio antigen mRNA to TriMix mRNA.

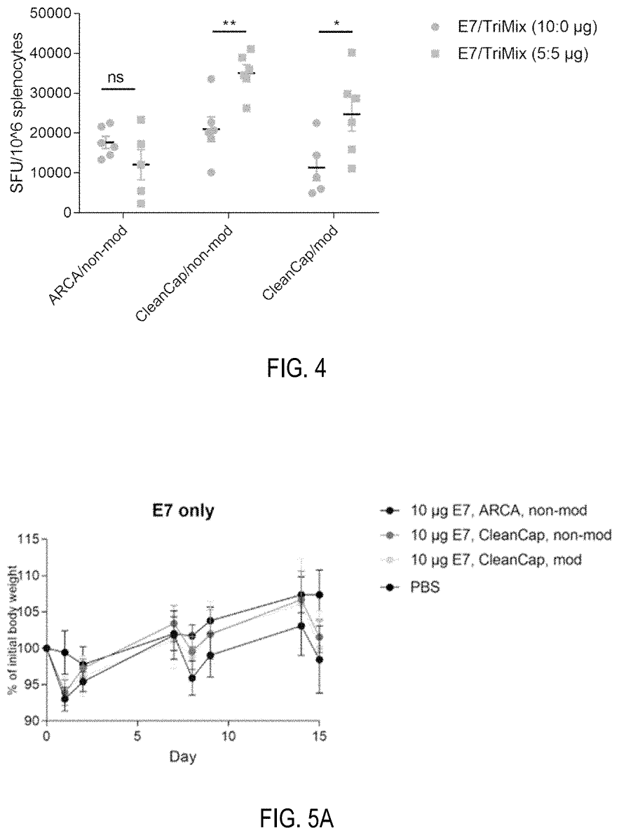

The magnitude and functionality of the E7-specific T cell response was further analyzed by IFN-γ ELISPOT on splenocytes obtained after the third immunization. Like what we observed in terms of E7-specific CD8 T cells circulating in blood, TriMix did not augment the number of IFN-γ producing splenocytes in case of ARCA, non-mod mRNA, yet TriMix did increase responses in case of CleanCap/non-mod mRNA and CleanCap/mod mRNAs (FIG. 4).

### Trimix Does Not Impact Weight Loss Nor Liver Damage

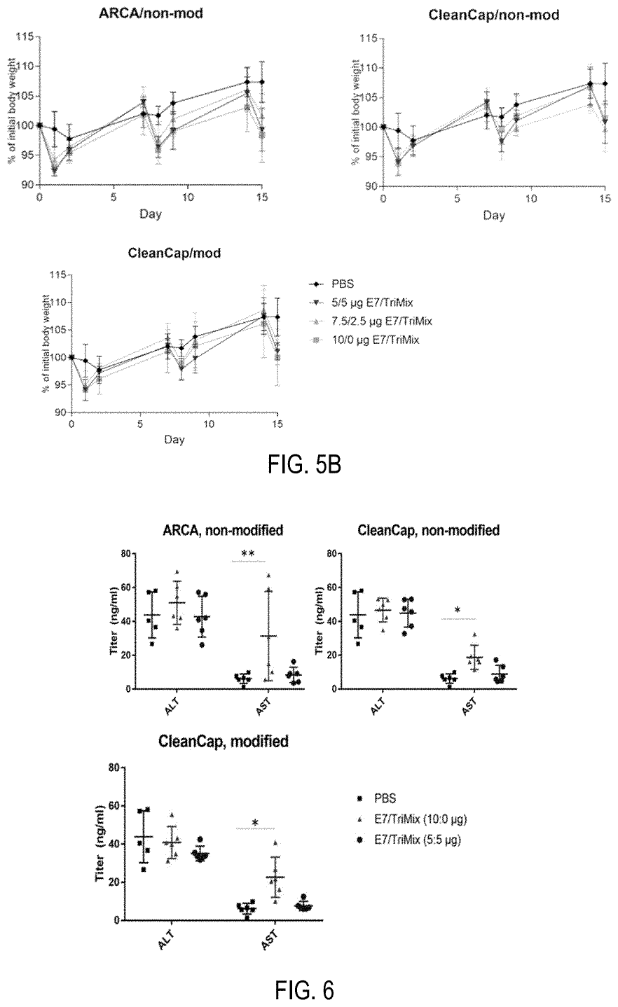

Weight loss and ALT/AST levels were measured as surrogate markers for toxicity and liver damage. As can be appreciated from FIGS. 5A and 5B, all LNPs induced transient weight loss of mice—most pronounced at 24 hrs post injection—followed by rapid recovery. The mRNA format itself appeared to have little impact on the extent of weight loss, although ARCA-capped mRNA tended to evoke the highest weight loss compared to PBS injected mice. For none of the mRNA formats addressed, TriMix exacerbated weight loss.

Finally, we assessed the impact of intravenous mRNA LNP vaccination on serum titers of the liver enzymes AST/ALT, used as surrogate markers for liver damage. AST/ALT levels were assessed by ELISA at one week after the third immunization. For none of the treatment groups, ALT/AST levels were strongly elevated. For E7/TriMix mRNA LNP treated mice, no statistically significant increases in ALT nor AST levels compared to PBS mice were measured, regardless of the mRNA format. Surprisingly, mice treated with E7-only mRNA LNPs did show low but significant upregulation in AST levels to PBS (FIG. 6).

### CONCLUSIONS

In this study, we have addressed the impact of mRNA format on the immunogenicity and inflammatory safety of antigen/TriMix mRNA vaccination using a previously optimized LNP formulation.

Replacement of ARCA by CleanCap resulted in a significant decrease in the systemic inflammatory response. The efficiency of co-transcriptional capping with ARCA is around 70%, leaving 30% of the generated mRNA with a 5′ triphosphate end, which is sensed by RIG-I. In addition, instead of the natural methylated Cap1 structure, ARCA introduces a non-methylated “Cap-0” structure, which again triggers various RNA sensors that drive inflammation. CleanCap (Cap-1) is incorporated with higher efficiency (>90%) and does introduce the natural Cap1 structure, hence lowering innate recognition and inflammation. Substitution of uridine by N1 methyluridine further lowered serum titers of IFN-a, IFN-g and IL-6, in line with previous reports demonstrating that N1 methylpseudo-uridine substitution lowers TLR7 and RIG-I activation. Importantly, for none of the E7/TriMix mRNA ratios and mRNA formats addressed, TriMix exacerbated systemic inflammatory responses.

In terms of magnitude and kinetics of the T cell response, no significant differences were observed between mRNA formats in the absence of TriMix. Yet, upon addition of TriMix, strong differences occurred between mRNA formats. In case of the high inflammatory ARCA, non-mod mRNA format, TriMix failed to further augment the magnitude of the T cell response. Nonetheless, in case of the low inflammatory mRNA formats CleanCap, non-mod and CleanCap, mod, TriMix exerted its immune-stimulatory functions and resulted in a strong increase in the magnitude of the E7-specific CD8 T cell response. Similar, TriMix mRNA also enhanced the numbers of IFN-γ secreting T cells in case of immunization with CleanCap, non-mod and CleanCap, mod mRNA yet not with ARCA, non-mod mRNA LNPs.

As CleanCap/mod mRNA LNPs with partial substitution of antigen mRNA by TriMix mRNA instigated the highest levels of T cell responses yet the lowest inflammatory response, we at this mRNA LNP format appears to display the best therapeutic index.

1-15. (canceled)

16. A combination comprising:
one or more mRNA molecules encoding all of the functional immunostimulatory proteins CD40L, CD70 and caTLR4; and
one or more mRNA molecules encoding one or more antigens or epitopes;
wherein at least one of the mRNA molecules is characterized in having a 5′ CAP-1 structure.

17. The combination of claim 16, wherein all of the mRNA molecules have a 5′ CAP-1 structure.

18. The combination of claim 16, wherein the mRNA molecules further comprise at least one modified nucleoside.

19. The combination of claim 18, wherein the at least one modified nucleoside is selected from the group consisting of pseudouridine, 5-methoxy-uridine, 5-methyl-cytidine, 2-thio-uridine, and N6-methyladenosine.

20. The combination of claim 18, wherein the at least one modified nucleoside is a pseudouridine selected from group consisting of 4-thio-pseudouridine, 2-thio-pseudouridine, 1-carboxymethyl-pseudouridine, 1-propynyl-pseudouridine, 1-taurinomethyl-pseudouridine, N1-methyl-pseudouridine, 4-thio-1-methyl-pseudouridine, 2-thio-1-methyl-pseudouridine, 1-methyl-1-deaza-pseudouridine, 2-thio-1-methyl-1-deaza-pseudouridine, dihydropseudouridine, 2-thio-dihydropseudouridine, 4-methoxy-pseudouridine, and 4-methoxy-2-thio-pseudouridine.

21. The combination of claim 18, wherein the at least one modified nucleoside is N1-methyl-pseudouridine.

22. The combination of claim 21, wherein about 100% of uridines in the mRNA molecules is replaced by N1-methyl-pseudouridine.

23. The combination of claim 16, wherein the one or more mRNA molecules encoding one or more antigens or epitopes encodes one or more cancer antigens.

24. A method for treating a cell proliferative disorder, the method comprising:
administering, to a subject in need thereof, a combination according to claim 16.

25. A pharmaceutical composition comprising the combination of claim 16 and at least one pharmaceutically acceptable agent.

26. The pharmaceutical composition of claim 25, wherein the combination is formulated in nanoparticles, such as lipid nanoparticles or polymeric nanoparticles.

27. The pharmaceutical composition of claim 25, wherein the composition is formulated for intravenous, intranodal, intratumoral, subcutaneous, intradermal or intramuscular formulation.

28. The pharmaceutical composition of claim 25, wherein the composition is in the form of a vaccine.

29. The pharmaceutical composition of claim 25, wherein the one or more mRNA molecules encoding one or more antigens or epitopes encodes one or more cancer antigens.

30. A method for treating a cell proliferative disorder, the method comprising:
administering, to a subject in need thereof, a pharmaceutical composition according to claim 25.


SEQUENCE LISTING (1 sequence)

SEQ ID NO: 1 - protein, 9 aa, Artificial Sequence
  E7 - Tetramer
RAHYNIVTF